(19) United States
(12) Patent Application Publication
(10) Pub. No.: US 2025/0154596 A1
(43) Pub. Date: May 15, 2025

(54) Method for Detecting Thyroid Tumor Based on Imprinted Gene Analysis

(71) Applicant: LISEN IMPRINTING DIAGNOSTICS, INC., Dover, DE (US)

(72) Inventors: Tong Cheng, Wuxi (CN); Ning Zhou, Wuxi (CN); Xing Li, Wuxi (CN)

(21) Appl. No.: 18/509,538

(22) Filed: Nov. 15, 2023

(51) Int. Cl. C12Q 1/6886; C12Q 1/6841; G16B 20/10; G16B 25/10

(52) U.S. Cl. CPC C12Q 1/6886; C12Q 1/6841; G16B 20/10; G16B 25/10; C12Q 2600/112; C12Q 2600/158

(57) ABSTRACT

A method for determining a level of malignancy of a thyroid tumor in a subject and treating the subject is provided. A test sample is obtained from the subject, in situ hybridization of probes designed based on the sequence of an intron of imprinted gene HM13 and imprinted gene SNRPN, respectively, with cells of the test sample is performed. The test sample having been subject to the hybridization is stained and the stained cells are observed under a microscope and the microscopic images of the stained test sample are analyzed to calculate parameters relating to the aberrant expression of the at least one imprinted gene. Such aberrant expression is then graded to determine the level of malignancy of a thyroid tumor.

### FIELD

The present invention relates to the field of biotechnology, such as the field of genetic diagnosis, specifically to a diagnostic method and uses thereof.

### BACKGROUND

According to international statistics on cancer, there are more than 8 million people who die of cancer every year worldwide. Therefore, early detection, diagnosis and treatment of cancer are particularly important. At the cellular level, cancer is produced by complex genetic and epigenetic changes that accumulate over time, ultimately leading to uncontrolled cell division.

Traditional pathology diagnosis for benign and malignant cells is based on cell size, morphology, invasiveness and their relationship to surrounding cellular tissues. The methods have great limitations on the early detection of cancer, so the cancer diagnosis method at the cellular and molecular level has once become a research hotspot.

At present, the determination of benign and malignant cancer and the determination of cancer type or cancer stage largely depend on the morphology of the cells observed by histopathological sections stained with hematoxylin and eosin. However, this method has its inherent limitations. First, the method cannot observe changes in the molecular level at the time of tumorigenesis, while molecular changes can provide a more accurate basis for pre-diagnosis and diagnosis. Secondly, the diagnosis of cell morphology is a subjective judgment, which in itself will cause inaccurate diagnosis; the error caused by such subjective judgment has a great impact on the diagnosis of early cancer, especially the sensitivity and accuracy of early diagnosis of cancer is critical for patients. Finally, some malignant tumor cells have little difference from benign tumor cells in morphology.

From the perspective of genetics, the development of tumors is a multi-gene process. Epigenetic modification has great impact for the occurrence, diagnosis and treatment of tumors, and has also been applied clinically. A large number of scientific studies have confirmed the correlation between re-expression of imprinted genes and cell carcinogenesis.

Thyroid nodules are common with a prevalence of about 70% on thyroid ultrasonography, and about 5% of them are malignant. Accurate assessment to distinguish malignant from benign thyroid nodules is critical for their appropriate clinical management. Although ultrasound imaging combined with fine-needle aspiration (FNA) biopsy is the diagnostic mainstay for thyroid nodules, about 20%-30% are diagnostically indeterminate with this approach. This diagnostic dilemma often causes confusion on how to treat a thyroid nodule clinically.

There are several thyroid diagnostic biomarker systems used variably around the world. These include mostly genetic alterations, gene expression, DNA methylation, and microRNAs, with each being associated with certain limitations. more effective biomarker-based diagnostic approach is needed for thyroid nodules.

Genomic imprinting is an epigenetic regulatory mechanism in mammalian embryo development and tumorigenesis. In normal somatic cells, paternal and maternal alleles of an imprinted gene are differentially methylated in an allele-specific manner, resulting in the silencing of one allele and activation of the other. In cancers, the normally silenced allele is often aberrantly activated in certain imprinted genes, resulting in the expressions of both alleles. This phenomenon is termed loss of imprinting (LOI), which is associated with various cancers. A nascent RNA in situ hybridization (ISH) method, targeting the short-lived introns to label and visualize transcription sites, has been widely used to study the transcriptional regulations of both imprinted and nonimprinted genes.

### SUMMARY

The present disclosure provides a method for determining a level of malignancy of a thyroid tumor in a subject and treating the subject, comprising:
obtaining a test sample from a subject;
performing in situ hybridization of a first probe designed based on the sequence of an intron of imprinted gene HM13 with a first plurality of cells of the test sample, and performing in situ hybridization of a second probe designed based on the sequence of an intron of imprinted gene SNRPN with a second plurality of cells of the test sample;
staining the first plurality of cells and the second plurality of cells having been subject to the hybridization with a staining chemical;
calculating a total expression, a biallelic expression, and a multiallelic expression for imprinted genes HM13 based on microscopic images of the stained first plurality of cells, and calculating a total expression, a biallelic expression, and a multiallelic expression for imprinted genes SNRPN based on microscopic images of the stained second plurality of cells, respectively;
grading the biallelic expression and multiallelic expression of each of the imprinted genes HM13 and SNRPN, deriving an overall gene score for each of the imprinted genes HM13 and SNRPN, and determining the level of malignancy of thyroid tumor of the subject based on a combination of the overall gene score for imprinted gene HM13 and imprinted gene SNRPN; and
treating the subject by administration of medication or other treatment in accordance with the determined level of malignancy of said thyroid tumor;
wherein the total expression (TE) of each of the imprinted genes HM13 and SNRPN, the biallelic expression (BAE) of each of the imprinted genes HM13 and SNRPN, and the multiallelic expression (MAE) of each of the imprinted genes HM13 and SNRPN are calculated by the following formula:

$$TE = ( b + c + d ) / ( a + b + c + d ) \times 100 \% ;$$

$$BAE = c / ( b + c + d ) \times 100 \% ;$$

$$MAE = d / ( b + c + d ) \times 100 \% ;$$

wherein, a represents the number of microscopically observed cells each of which has no mark in the nucleus of the cell after the staining, b represents the number of microscopically observed cells for each of which there is one red/brown mark in the nucleus of the cell after the staining, c represents the number of microscopically observed cells for each of which there are two red/brown marks in the nucleus of the cell after the staining, and d represents the number of microscopically observed cells for each of which there are more than two red/brown markers in the nucleus of the cell after the staining.

In some embodiments, the staining chemical comprises hematoxylin. In some embodiments, the staining chemical is H&E stain.

In some embodiments, the probe for an imprinted gene HM13 and SNRPN is designed based on a sequence within an intron of each imprinted gene as a template. As such, the probe comprises a sequence complementary to the sequence within the intron of the imprinted gene.

The in situ hybridization can be RNAscope in situ hybridization. The RNAscope in situ hybridization can be performed by using singleplex or multiplex chromogenic assay kit or singleplex or multiplex fluorescence assay kit, preferably singleplex red/brown chromogenic assay kit or multiplex fluorescence assay kit. The multiplex chromogenic assay kit or multiplex fluorescence assay kit can include two or more channels of chromogenic assay kit or fluorescence assay kit. The two channels chromogenic assay kit or multiple channels fluorescence assay kit can use two imprinted gene probes to detect the expression of an imprinted gene and another gene or even the expression of multiple imprinted genes and nonimprinted genes.

In some embodiments, the biallelic expression of each of the imprinted genes is classified into 5 grades, and the multiallelic expression of each of the imprinted genes is classified into 5 grades.

In some embodiments, if the total expression of HM13 is <a first predetermined threshold T_{Z19-T1}, the biallelic expression of HM13 and the multiallelic expression of HM13 are both classified to be Grade 0; if the total expression is >=T_{Z19-T1}, the biallelic expression of HM13 is classified into 5 grades according to four thresholds T_{Z19-B1}, T_{Z19-B2}, T_{Z19-B3}, T_{Z19-B4}, and the multiallelic expression of HM13 is also separately classified into the 5 grades according to four thresholds T_{Z19-M1}, T_{Z19-M2}, T_{Z19-M3}, T_{Z19-M4}: Grade 0: the biallelic expression of HM13 is <T_{Z19-B1} and the multiallelic expression of HM13 is <T_{Z19-M1}; Grade 1: the biallelic expression of HM13 is >=T_{Z19-B1} and <T_{Z19-B2}, and the multiallelic expression of HM13 is >=T_{Z19-M1} and <T_{Z19-M2}; Grade 2: the biallelic expression of HM13 is >=T_{Z19-B2} and <T_{Z19-B3}, and the multiallelic expression of HM13 is >=T_{Z19-M2} and <T_{Z19-M3}; Grade 3: the biallelic expression of HM13 is >=T_{Z19-B3} and <T_{Z19-B4}, and the multiallelic expression of HM13 is >=T_{Z19-M3} and <T_{Z19-M4}; Grade 4: the biallelic expression of HM13 is >=T_{Z19-B4} and the multiallelic expression of HM13 is >=T_{Z19-M4}. In some examples, these thresholds are: T_{Z19-T1}=11.24%; T_{Z19-B1}, T_{Z19-B2}, T_{Z19-B3}, T_{Z19-B4}=12.39%, 20.07%, 26.28%, and 30.00%, respectively; and T_{Z19-M1}, T_{Z19-M2}, T_{Z19-M3}, T_{Z19-M4}=1.43%, 3.39%, 6.31%, and 9.42%, respectively, as shown in Table 3 (FNA model).

In some embodiments, if the total expression of SNRPN is <a second predetermined threshold T_{Z16-T1}, the biallelic expression of SNRPN and the multiallelic expression of SNRPN are both classified to be Grade 0; if the total expression is >=T_{Z16-T1}, the biallelic expression of SNRPN is classified into 5 grades according to four thresholds T_{Z16-B1}, T_{Z16-B2}, T_{Z16-B3}, T_{Z16-B4}, and the multiallelic expression of SNRPN is also separately classified into 5 grades according to four thresholds T_{Z16-M1}, T_{Z16-M2}, T_{Z16-M3}, T_{Z16-M4}: Grade 0: the biallelic expression of SNRPN is <T_{Z16-B1} and the multiallelic expression of SNRPN is <T_{Z16-M1}; Grade 1: the biallelic expression of SNRPN is >=T_{Z16-B1} and <T_{Z16-B2}, and the multiallelic expression of SNRPN is >=T_{Z16-M1} and <T_{Z16-M2}; Grade 2: the biallelic expression of SNRPN is >=T_{Z16-B2} and <T_{Z16-B3}, and the multiallelic expression of SNRPN is >=T_{Z16-M2} and <T_{Z16-M3}; Grade 3: the biallelic expression of SNRPN is >=T_{Z16-B3} and <T_{Z16-B4}, and the multiallelic expression of SNRPN is >=T_{Z16-M3} and <T_{Z16-M4}; Grade 4: the biallelic expression of SNRPN is >=T_{Z16-B4} and the multiallelic expression of SNRPN is >=T_{Z16-M4}. In some examples, these thresholds are: T_{Z16-T1}=17.38%; T_{Z16-B1}, T_{Z16-B2}, T_{Z16-B3}, T_{Z16-B4}=15.29%, 20.25%, 26.41%, and 30.77%, respectively; and T_{Z16-M1}, T_{Z16-M2}, T_{Z16-M3}, T_{Z16-M4}=1.44%, 3.41%, 5.54%, 9.02%, respectively, as shown in Table 3 (FNA model).

TABLE 3
Thresholds for FIG. 5A

| | QCIGISH FNA Model | | | | | |
|---|---|---|---|---|---|---|
| Model | [symbol] SNRPN | | | HM13 | | |
| Thresholds | BAE_{T} | MAE_{T} | TE_{T} | BAE_{T} | MAE_{T} | TE_{T} |
| T = 1 | 16.29% | 1.44% | 17.38% | 12.39% | 1.43% | 11.24% |
| T = 2 | 20.25% | 3.41% | N/A | 20.07% | 3.39% | N/A |
| T = 3 | 26.41% | 5.54% | N/A | 26.28% | 6.31% | N/A |
| T = 4 | 30.77% | 9.02% | N/A | 3 [symbol] .00% | 9.42% | N/A |

[symbol]

indicates data missing or illegible when filed

In some embodiments, wherein the level of malignancy of the thyroid tumor to be determined is classified as 5 categories indicated by integers 0, 1, 2, 3, and 4, wherein greater integers indicate greater malignancy risks, with 0 indicating benign thyroid tumor, and 4 indicating thyroid tumor with the highest malignancy. In some embodiments, the first two categories indicated by 0 and 1 are considered benign, and the second three categories indicated by 2, 3, and 4 are considered progressively more severe malignancy.

In some embodiments, an overall gene score can be derived for each imprinted gene according to the grade of the gene's biallelic expression and the grade of the gene's multiallelic expression. For example, as illustrated in FIG. 5B, each of the overall HM13 gene score and overall SNRPN gene score can be computed as follows: Score 0: the grade of the gene's biallelic expression and the grade of the gene's multiallelic expression are both grade 0; Score 1: the grade of the gene's multiallelic expression is grade 0 and the grade of the gene's biallelic expression is >=grade 1, or the grade of the gene's multiallelic expression is grade 1 and the grade of the gene's biallelic expression is <=grade 1; Score 2: the grade of the gene's multiallelic expression is grade 1 and the grade of the gene's biallelic expression is >=grade 2, or the grade of the gene's multiallelic expression is grade 2 and the grade of the gene's biallelic expression is <=grade 2; Score 3: the grade of the gene's multiallelic expression is grade 2 and the grade of the gene's biallelic expression is >=grade 3, or the grade of the gene's multiallelic expression is grade 3 and the grade of the gene's biallelic expression is <=grade 3; Score 4: the grade of the gene's multiallelic expression is grade 3 and the grade of the gene's biallelic expression is grade 4, or the grade of the gene's multiallelic expression is grade 4 and the grade of the gene's biallelic expression is <=grade 4.

In some embodiments, the level of malignancy of the thyroid tumor is determined based on an evaluation of the derived overall SNRPN gene score and the derived overall HM13 gene score. For example, and as illustrated in FIG. 5C, the evaluation can comprise: calculating a weighted sum of the derived overall SNRPN gene score and the derived overall HM13 gene score; and comparing the weighted sum against predetermined four numerical thresholds defining five numerical ranges and determining which of the five numerical ranges the weighted sum falls into. In some embodiments, a greater weight is assigned to the derived HM13 gene score than to the derived SNRPN gene score. In some examples, a weight of 0.7 is assigned to the overall HM13 gene score whereas a weight of 0.3 is assigned to the overall SNRPN gene score. In some embodiments, the four numerical thresholds are 0.5, 1.5, 2.5, and 3.5, respectively.

After reading and understanding the accompanying drawings and detailed description, other aspects of the present disclosure will be understood.

### BRIEF DESCRIPTION OF THE DRAWINGS

The drawings are used to provide a further understanding of the embodiments of the invention, and are to be construed as a part of the description of the invention.

FIG. 1 shows the study design and workflow diagram for Example 1. FFPE, formalin-fixed paraffin-embedded; FNA, fine-needle aspiration; QCIGISH, quantitative chromogenic imprinted gene in situ hybridization.

FIGS. 2A and 2B illustrates the principle of quantitative chromogenic imprinted gene in situ hybridization (QCIGISH)-visualization, quantification, and pathologic confirmation of the allelic expression status of imprinted genes. (FIG. 2A) Conceptual framework of QCIGISH. Shown is a QCIGISH-stained tissue section of a case of PTC. Blue components in the image are cell nuclei stained using hematoxylin. The red vertical lines on the chromosome map indicate the gene loci. The blue horizontal lines under the intron/exon map of premRNAs indicate the targeted introns of the in situ hybridization probes. (FIG. 2B) Pathologic confirmation of the QCIGISH results using simultaneous hematoxylin and cosin staining examination of a case of PTC. The low-magnification image was captured at a particular tumor region showing both malignant (lower subregion) and benign (upper subregion) morphologic characteristics. BAE, biallelic expression; MAE, multiallelic expression; ncRNA, noncoding RNA; PTC, papillary thyroid carcinoma; QCIGISH, quantitative chromogenic imprinted gene in situ hybridization; TE, total expression.

FIGS. 3A-3G show performance of QCIGISH test in the blinded prospective validation cohort. (FIG. 3A) Distribution of histopathologically benign and malignant cases in different ultrasound imaging categories. (FIG. 3B) Distribution of histopathologically benign and malignant cases in different Bethesda cytopathology categories. (FIG. 3C) Sensitivity, specificity, PPV, and NPV of the QCIGISH test in the blinded prospective validation study. Distribution of histopathologically benign and malignant cases in QCIGISH-negative and QCIGISH-positive groups for (FIG. 3D) Bethesda II, (FIG. 3E) Bethesda III and IV, (FIG. 3F) Bethesda V, and (FIG. 3G) Bethesda IV categories. Histopathologic benign and malignant diagnoses were confirmed by postsurgical thyroid examination in all cases. ACR TI-RADS, American College of Radiology Thyroid Imaging, Reporting, and Data System; NED, no evidence of disease; NPV, negative predictive value; PPV, positive predictive value; QCIGISH, quantitative chromogenic imprinted gene in situ hybridization.

FIGS. 4A-4C show final thresholds for grading the expression of the two imprinted genes HM13 and SNRPN in Example 1.

FIGS. 5A-5C show flow diagrams of the grading model for determining the malignancy level of thyroid cancer in a patient in Example 1.

### DETAILED DESCRIPTION

The examples herein will be described in detail below with reference to the drawings.

In general, the present disclosure provides a method to analyze a tumor obtained as a biopsy sample of a patient, determines the level of malignancy of the tumor, thereby providing a basis for surgery and precise treatment. As used herein, the level of malignancy including benign tumor as well as moderate and more advanced tumor.

The present disclosure provides a method for determining a level of malignancy of a thyroid tumor in a subject (such as a human) and treating the subject, comprising the following. A test sample from the subject is obtained, e.g., by fine needle aspiration. Thus the test sample may be a needle biopsy sample, e.g., from the thyroid of the subject. The test sample can be divided into several portions, for example, a first portion which includes a first plurality of cells and a second portion which includes a second plurality of cells. A first probe (nucleic acid probe) is used to perform in situ hybridization with the first portion of the test sample (or the first plurality of cells), where the first probe specifically hybridizes to imprinted gene HM13, e.g., the first probe can be designed based on a sequence within an intron of the imprinted gene HM13. A second probe (nucleic acid probe) is used to perform in situ hybridization with the second portion of the test sample (or the second plurality of cells), where the second probe specifically hybridizes to imprinted gene SNRPN, e.g., the second probe can be designed based on a sequence within an intron of the imprinted gene SNRPN.

Then the first plurality of cells and the second plurality of cells having been subject to the hybridization are stained with a staining chemical.

The stained first and second plurality of cells are detected or observed separately under one or more microscopes to analyze the staining result. There are four different types of microscopic images of the cells after staining with respect to each of the imprinted gene HM13 and imprinted gene SNRPN: (1) cells that have no mark in their nuclei after the staining, (2) cells that have one red/brown mark in their nuclei after the staining, (3) cells that have two red/brown marks in their nuclei after the staining, and (4) cells that have more than two red/brown markers in their nuclei after the staining. These different types of cells can be detected either by human or by computer image processing techniques or algorithms on the digital microscopic images of the cells.

A total expression (TE), a biallelic expression (BAE), and a multiallelic expression (MAE) for imprinted genes HM13 can be calculated based on the microscopic images of the stained first plurality of cells, by the following formula:

$$TE = ( b + c + d ) / ( a + b + c + d ) \times 100 \% ;$$

$$BAE = c / ( b + c + d ) \times 100 \% ;$$

$$MAE = d / ( b + c + d ) \times 100 \% ;$$

wherein, a represents the number of microscopically observed cells each of which has no mark in the nucleus of the cell after the staining, b represents the number of microscopically observed cells for each of which there is one red/brown mark in the nucleus of the cell after the staining, c represents the number of microscopically observed cells for each of which there are two red/brown marks in the nucleus of the cell after the staining, and d represents the number of microscopically observed cells for each of which there are more than two red/brown markers in the nucleus of the cell after the staining. a+b+c+d would be the total number of cells selected/analyzed under the microscope, which would be fewer than the number of the first plurality of cells. However, for reliable results, at least 1,000 of randomly selected cells among the stained first plurality of cells should be examined to determine their expression status with respect to the imprinted gene HM13.

Similarly, TE, BAE, and MAE can also be calculated for imprinted gene SNRPN based on the microscopic images of the stained second plurality of cells by the same formula above, i.e.,

$$TE = ( b + c + d ) / ( a + b + c + d ) \times 100 \% ;$$

$$BAE = c / ( b + c + d ) \times 100 \% ;$$

$$MAE = d / ( b + c + d ) \times 100 \% ;$$

wherein, a represents the number of microscopically observed cells each of which has no mark in the nucleus of the cell after the staining, b represents the number of microscopically observed cells for each of which there is one red/brown mark in the nucleus of the cell after the staining, c represents the number of microscopically observed cells for each of which there are two red/brown marks in the nucleus of the cell after the staining, and d represents the number of microscopically observed cells for each of which there are more than two red/brown markers in the nucleus of the cell after the staining. a+b+c+d would be the total number of cells selected/analyzed under the microscope, which would be fewer than the number of the second plurality of cells. However, for reliable results, at least 1,000 of randomly selected cells among the stained second plurality of cells should be examined to determine their expression status with respect to the imprinted gene SNRPN.

Then, the TE, BAE, and MAE of each of the imprinted genes HM13 and SNRPN can be graded. An overall gene score for each of the imprinted genes HM13 and SNRPN can be derived, based on which the level of malignancy of the thyroid tumor can be determined. The subject can then be treated by administration of medication or other treatment in accordance with the determined level of malignancy of the thyroid tumor;

In some embodiments, the probe for an imprinted gene HM13 and SNRPN is designed based on a sequence within an intron of each imprinted gene as a template. As such, the probe comprises a sequence complementary to the sequence within the intron of the imprinted gene.

In some embodiments, the staining chemical comprises hematoxylin. In some embodiments, the staining chemical is H&E stain.

The in situ hybridization can be RNAscope in situ hybridization. The RNAscope in situ hybridization can be performed by using singleplex or multiplex chromogenic assay kit or singleplex or multiplex fluorescence assay kit, preferably singleplex red/brown chromogenic assay kit or multiplex fluorescence assay kit. The multiplex chromogenic assay kit or multiplex fluorescence assay kit can include two or more channels of chromogenic assay kit or fluorescence assay kit. The two channels chromogenic assay kit or multiple channels fluorescence assay kit can use two imprinted gene probes to detect the expression of multiple imprinted genes.

In some embodiments, the biallelic expression of each of the imprinted genes is classified into 5 grades, and the multiallelic expression of each of the imprinted genes is classified into 5 grades.

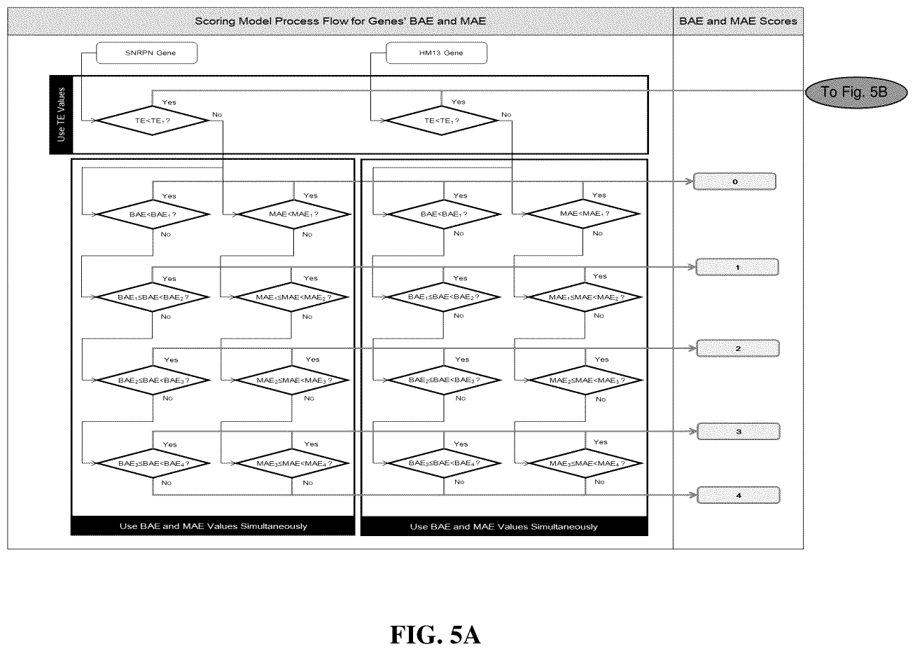

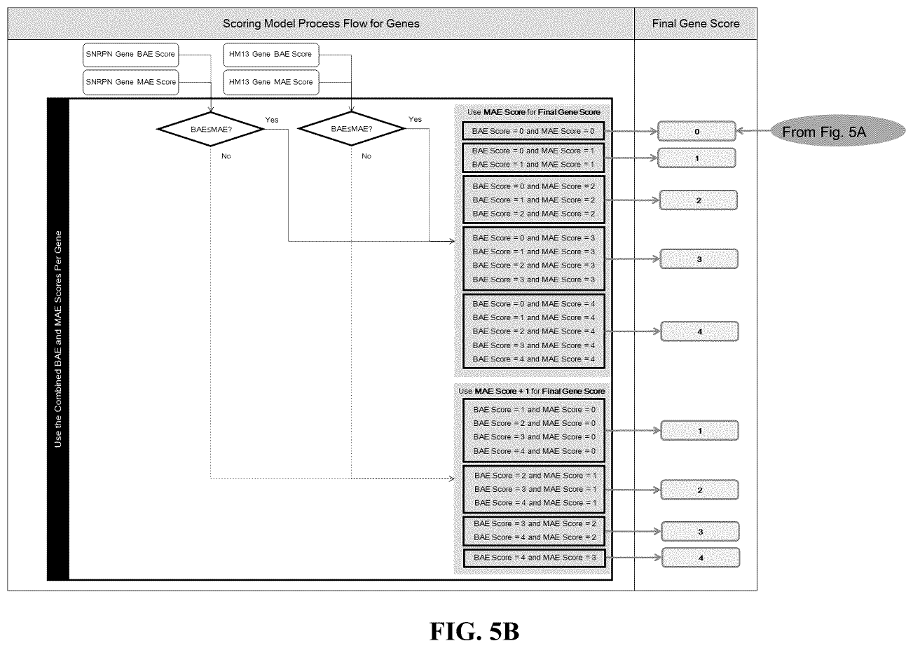

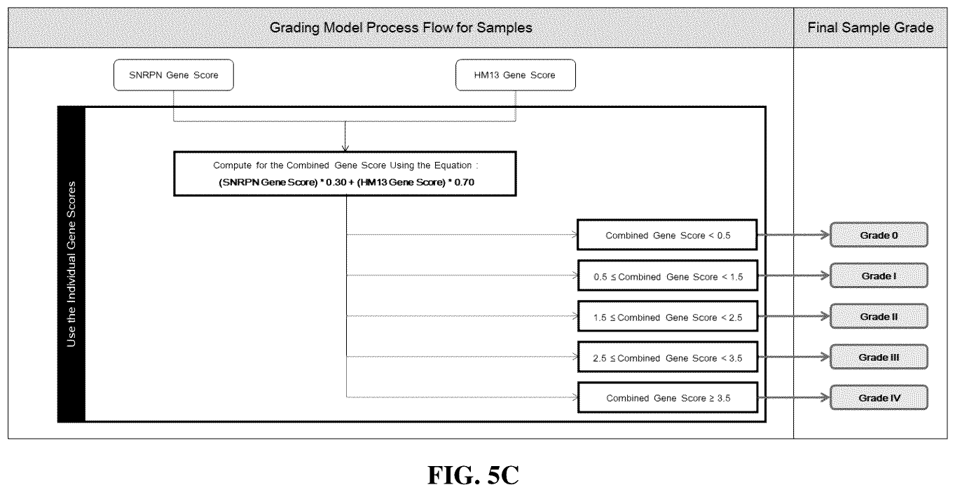

In some embodiments, if the total expression of HM13 is <a first predetermined threshold T_{Z19-T1}, the biallelic expression of HM13 and the multiallelic expression of HM13 are both determined to be Grade 0; if the total expression is >=T_{Z19-T1}, the biallelic expression of HM13 is classified into 5 grades according to four thresholds T_{Z19-B1}, T_{Z19-B2}, T_{Z19-B3}, T_{Z19-B4}, and the multiallelic expression of HM13 is also classified into 5 grades according to four thresholds T_{Z19-M1}, T_{Z19-M2}, T_{Z19-M3}, T_{Z19-M4}: Grade 0: the biallelic expression of HM13 is <T_{Z19-B1} and the multiallelic expression of HM13 is <T_{Z19-M1}; Grade 1: the biallelic expression of HM13 is >=T_{Z19-B1} and <T_{Z19-B2}, and the multiallelic expression of HM13 is >=T_{Z19-M1} and <T_{Z19-M2}; Grade 2: the biallelic expression of HM13 is >=T_{Z19-B2} and <T_{Z19-B3}, and the multiallelic expression of HM13 is >=T_{Z19-M2} and <T_{Z19-M3}; Grade 3: the biallelic expression of HM13 is >=T_{Z19-B3} and <T_{Z19-B4}, and the multiallelic expression of HM13 is >=T_{Z19-M3} and <T_{Z19-M4}; Grade 4: the biallelic expression of HM13 is >=T_{Z19-B4} and the multiallelic expression of HM13 is >=T_{Z19-M4}. In some examples, these thresholds are: T_{Z19-T1}=11.24%; T_{Z19-B1}, T_{Z19-B2}, T_{Z19-B3}, T_{Z19-B4}=12.39%, 20.07%, 26.28%, and 30.00%, respectively; and T_{Z19-M1}, T_{Z19-M2}, T_{Z19-M3}, T_{Z19-M4}=1.43%, 3.39%, 6.31%, and 9.42%, respectively, as shown in FIG. 5A (FNA model).

In some embodiments, if the total expression of SNRPN is <a second predetermined threshold T_{Z16-T1}, the biallelic expression of SNRPN and the multiallelic expression of SNRPN are both determined to be Grade 0; if the total expression is >=T_{Z16-T1}, the biallelic expression of SNRPN is classified into 5 grades according to four thresholds T_{Z16-B1}, T_{Z16-B2}, T_{Z16-B3}, T_{Z16-B4}, and the multiallelic expression of SNRPN is also classified into 5 grades according to four thresholds T_{Z16-M1}, T_{Z16-M2}, T_{Z16-M3}, T_{Z16-M4}: Grade 0: the biallelic expression of SNRPN is <T_{Z16-B1} and the multiallelic expression of SNRPN is <T_{Z16-M1}; Grade 1: the biallelic expression of SNRPN is >=T_{Z16-B1} and <T_{Z16-B2}, and the multiallelic expression of SNRPN is >=T_{Z16-M1} and <T_{Z16-M2}; Grade 2: the biallelic expression of SNRPN is >=T_{Z16-B2} and <T_{Z16-B3}, and the multiallelic expression of SNRPN is >=T_{Z16-M2} and <T_{Z16-M3}; Grade 3: the biallelic expression of SNRPN is >=T_{Z16-B3} and <T_{Z16-B4}, and the multiallelic expression of SNRPN is >=T_{Z16-M3} and <T_{Z16-M4}; Grade 4: the biallelic expression of SNRPN is >=T_{Z16-B4} and the multiallelic expression of SNRPN is >=T_{Z16-M4}. In some examples, these thresholds are: T_{Z16-T1}=17.38%; T_{Z16-B1}, T_{Z16-B2}, T_{Z16-B3}, T_{Z16-B4}=15.29%, 20.25%, 26.41%, and 30.77%, respectively; and T_{Z16-M1}, T_{Z16-M2}, T_{Z16-M3}, T_{Z16-M4}=1.44%, 3.41%, 5.54%, 9.02%, respectively, as shown in FIG. 5A (FNA model).

In some embodiments, wherein the level of malignancy of the thyroid tumor to be determined is classified as 5 categories indicated by integers 0, 1, 2, 3, and 4, wherein greater integers indicate greater malignancy risks, with 0 indicating benign thyroid tumor, and 4 indicating thyroid tumor with the highest malignancy. In some embodiments, the first two categories indicated by 0 and 1 are considered benign, and the second three categories indicated by 2, 3, and 4 are considered progressively more severe malignancy.

In some embodiments, an overall gene score can be derived for each imprinted gene according to the grade of the gene's biallelic expression and the grade of the gene's multiallelic expression. For example, as illustrated in FIG. 5B, each of the overall HM13 gene score and overall SNRPN gene score can be computed as follows: Score 0: if the grade of the gene's biallelic expression and the grade of the gene's multiallelic expression are both grade 0; Score 1: if the grade of the gene's multiallelic expression is grade 0 and the grade of the gene's biallelic expression is >=grade 1, or if the grade of the gene's multiallelic expression is grade 1 and the grade of the gene's biallelic expression is <=grade 1; Score 2: if the grade of the gene's multiallelic expression is grade 1 and the grade of the gene's biallelic expression is >=grade 2, or if the grade of the gene's multiallelic expression is grade 2 and the grade of the gene's biallelic expression is <=grade 2; Score 3: if the grade of the gene's multiallelic expression is grade 2 and the grade of the gene's biallelic expression is >=grade 3, or if the grade of the gene's multiallelic expression is grade 3 and the grade of the gene's biallelic expression is <=grade 3; Score 4: if the grade of the gene's multiallelic expression is grade 3 and the grade of the gene's biallelic expression is grade 4, or if the grade of the gene's multiallelic expression is grade 4 and the grade of the gene's biallelic expression is <=grade 4.

In some embodiments, the level of malignancy of the thyroid tumor is determined based on an evaluation of the derived overall SNRPN gene score and the derived overall HM13 gene score. For example, and as illustrated in FIG. 5C, the evaluation can comprise: calculating a weighted sum of the derived overall SNRPN gene score and the derived overall HM13 gene score; and comparing the weighted sum against predetermined four numerical thresholds defining five numerical ranges and determining which of the five numerical ranges the weighted sum falls into. In some embodiments, a greater weight is assigned to the derived HM13 gene score than to the derived SNRPN gene score. In some examples, a weight of 0.7 is assigned to the overall HM13 gene score whereas a weight of 0.3 is assigned to the overall SNRPN gene score. In some embodiments, the four numerical thresholds are 0.5, 1.5, 2.5, and 3.5, respectively.

After the level of malignancy of the thyroid tumor is determined by the disclosed method, a further medical intervention, such as a surgery, medication, radiotherapy, etc., appropriate for the thyroid tumor can be considered and executed to treat the tumor. For example, microwave ablation and radiofrequency ablation can be performed on benign thyroid tumors to avoid more invasive surgery and to retain functioning thyroid for hormone secretion; partial or total thyroidectomy can be performed on malignant thyroid tumors, and radiotherapy including Iodine-131 and chemotherapy including Doxorubicin, Cabozantinib, Lenvatinib or Sorafenib can be further used to kill the cancer cells retained in the cancer locus and spread to bloodstream or lymphatic system.

### Example 1

This example provides a method for determining thyroid cancer using an imprinted gene. The details are provided in Xu at al., “High Diagnostic Accuracy of Epigenetic Imprinting Biomarkers in Thyroid Nodules”, DOI: 10.1200/JCO.22.00232 Journal of Clinical Oncology 41, no. 6 (Feb. 20, 2023) 1296-1306, the disclosure of which (including Data Supplement thereof) is incorporated by reference in its entirety herein.

Generally, quantitative chromogenic imprinted gene in situ hybridization (QCIGISH) technique was used to investigate the diagnostic value of the expression status of three imprinted genes [guanine nucleotide-binding protein, alpha-stimulating complex locus (GNAS), growth factor receptor-bound protein (GRB10), and small nuclear ribonucleoprotein polypeptide N (SNRPN)] and the imprinted gene minor histocompatibility antigen H13 (HM13) on presurgical thyroid FNA specimens of thyroid nodules and matched histopathologic tissues. The combination of SNRPN and HM13 was found to be particularly efficient for developing an accurate diagnostic grading model for thyroid nodules.

Overall, a total of 550 patients with fine-needle aspiration (FNA)-evaluated and histopathologically confirmed thyroid nodules were consecutively recruited from eight medical centers. Quantitative chromogenic imprinted gene in situ hybridization (QCIGISH) was used to assess the allelic expression of imprinted genes SNRPN and HM13, on the basis of which a diagnostic grading model for thyroid nodules was developed. The model was retrospectively trained on 124 postsurgical thyroid samples, optimized on 32 presurgical FNA samples, and prospectively validated on 394 presurgical FNA samples. Blinded central review-based cytopathologic and histopathologic diagnoses were used as the reference standard. For thyroid malignancy, the QCIGISH test achieved an overall diagnostic sensitivity of 100% (277/277), a specificity of 91.5% (107/117; 95% CI, 86.4 to 96.5), a positive predictive value (PPV) of 96.5% (95% CI, 94.4 to 98.6), and a negative predictive value (NPV) of 100% in the prospective validation, with a diagnostic accuracy of 97.5% (384/394; 95% CI, 95.9 to 99.0). QCIGISH demonstrated a PPV of 97.8% (95% CI, 94.7 to 100) and NPV of 100%, with a diagnostic accuracy of 98.2% (111/113; 95% CI, 95.8 to 100), for indeterminate Bethesda III-V thyroid nodules. QCIGISH demonstrated a PPV of 96.6% (95% CI, 91.9 to 100) and a NPV of 100%, with a diagnostic accuracy of 97.5% (79/81; 95% CI, 94.2 to 100), for Bethesda III-IV. For Bethesda VI, QCIGISH showed a 100% (184/184) accuracy.

### Study Design and Participants

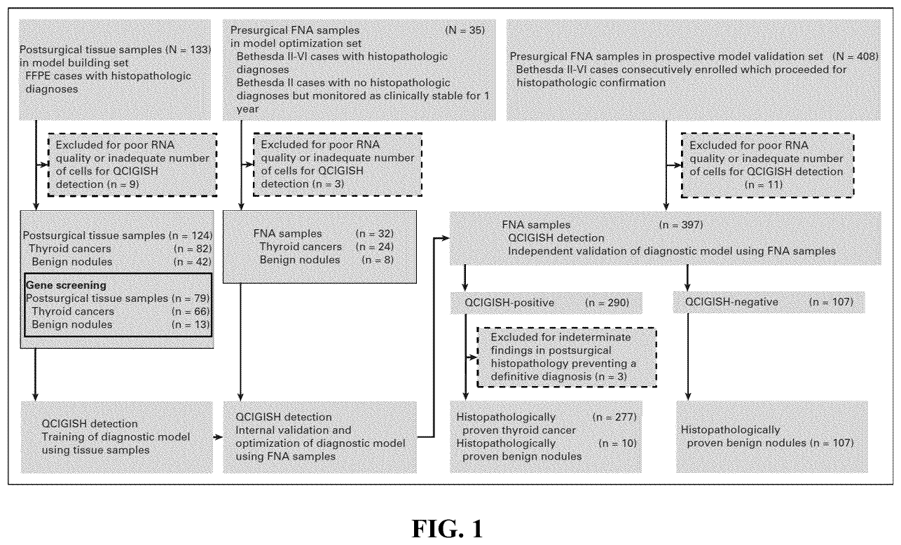

Patients with ultrasound-detected thyroid nodules recommended to have FNA evaluations were recruited from eight medical centers, including Shanghai Tenth People's Hospital of Tongji University School of Medicine, Jiangyuan Hospital affiliated to Jiangsu Institute of Nuclear Medicine, Taizhou People's Hospital, Taizhou Third People's Hospital, Shengjing Hospital of China Medical University, Nanjing First Hospital, Cancer Hospital of the University of Chinese Academy of Sciences, and Henan Cancer Hospital. Patients were divided into three groups for different test purposes as illustrated in FIG. 1.

Thyroid ultrasound examination and fine-needle aspiration biopsy were performed to nodules with relatively high-grade ACR TI-RADS categories and other clinical risk indications. All FNAs were conducted by experienced radiologists under ultrasound guidance. For each thyroid nodule, the FNA specimen was expelled onto a glass slide and mixed with a pipette. An amount of 5 μl of the specimen was extracted and fixed in 10% formalin neutral buffer for QCIGISH detection. The remaining specimen was smeared onto glass slides and fixed with 95% alcohol for cytopathology analysis. The FNA specimens were immediately analyzed by pathologists and reported according to the Bethesda cytology classification system Specimens classified under Bethesda II (benign cytology results from ultrasound-guided FNA), Bethesda III (atypia of undetermined significance or follicular lesion of undetermined significance), Bethesda IV (follicular neoplasm or suspicious for a follicular neoplasm), Bethesda V (suspicious for malignancy), or Bethesda VI (malignant) whose diagnoses were eventually histopathologically confirmed as benign or malignant were included in the model optimization and validation sets. Early cases were collected and used for model optimization, after which cases were consecutively and prospectively collected for validation. Some cases of Bethesda II thyroid nodules that did not undergo surgery but had clinical stability after at least one year of clinical follow-up were also included in the benign group of thyroid nodules for the model optimization. These patients with Bethesda II thyroid nodules were treated following the American Association of Endocrine Surgeons Guidelines stating that these cases can be safely observed and surgery might be considered for cases associated with local compressive symptoms due to large nodule size or the preference of the patient.

Clinical characteristics of the subjects are shown in Table 1. We used 124 formalin-fixed paraffin-embedded (FFPE) postsurgical thyroid specimens for initial diagnostic model building (FIG. 1). These consisted of 42 benign nodules and 82 malignant nodules (68 papillary thyroid carcinoma [PTC] and 14 follicular thyroid carcinoma [FTC]). We used 32 presurgical thyroid FNA specimens for model optimization, including eight benign nodules with Bethesda II cytologic diagnosis and 24 malignant nodules (21 PTC and three FTC, all pathologically confirmed; FIG. 1). A total of 408 cases of thyroid nodules (ACR TI-RADS category 3-5) were consecutively recruited for prospective model validation of the QCIGISH test (FIG. 1); they all had postsurgical histopathology diagnoses. Among these, 11 cases of poor-quality FNA specimen and three cases with indeterminate postsurgical histopathology were excluded. The operated benign (Bethesda II) nodules were so treated for clinical symptoms or patient's preference per standard clinical practice guidelines.23 A total of 394 FNA samples that had postsurgical histopathology were finally included for the prospective validation study. These included histopathologically confirmed 117 benign nodules and 277 malignant nodules (270 PTC, five FTC, one poorly differentiated thyroid cancer, and one medullary thyroid carcinoma). The QCIGISH testing on FNA specimen was performed in a blinded manner: persons conducting the QCIGISH test were blinded to the clinical, cytologic, and histopathologic diagnoses. The FNA samples from each patient were subjected to simultaneous Bethesda cytology classification and QCIGISH testing with the specimens analyzed and blindly scored according to the diagnostic QCIGISH grading criteria.

TABLE 1
Clinical Characteristics of the Study Subjects

| | Patient Cohorts | | |
|---|---|---|---|
| | Model | Model | Model |
| | Building | Optimization | Validation |
| | (tissue | (FNA | (FNA |
| Patient | specimens; | specimens; | specimens; |
| Characteristic | n = 124) | n = 24 + 8) [symbol] | n = 394) [symbol] |
| Patient sex | | | |
| Male, No. (%) | 34 (27.4) | 3 (12.5) | 100 (25.5) |
| Female, No. (%) | 90 (72.6) | 21 (87.5) | 292 (74. [symbol] ) |
| Patient age, years | | | |
| Median | 46 | 42 | 47 |
| IQR | 36-55 | 35-57 | 36-58 |
| Nodule size, cm | | | |
| Median | 1.3 | 1.5 | 1.2 |
| IQR | 0.7-2.5 | 1. [symbol] -3.0 | 0.8-2.3 |
| ACR TI-RADS category | | | |
| 3 | — | — | 76 |
| 4 | — | — | 59 |
| 5 | — | — | 259 |
| Bethesda classification | | | |
| II | — | 0 [symbol] | 97 |
| III | — | 2 | 51 |
| IV | — | 2 | 30 |
| V | — | 4 | 32 |
| VI | — | 16 | 1 [symbol] 4 |
| Histopathologic diagnosis | | | |
| Benign nodules | 42 | 0 [symbol] | 117 |
| NG | 17 | | 71 |
| FTA | 14 | | 22 |
| Adenoma | 5 | | 1 |
| Adenomatous goiter | 3 | | 10 |
| HT | 3 | | 6 |
| Thyroiditis | | | 5 |
| Cystic nodule | | | 1 |
| Benign [symbol] e | | | 1 |
| PTC | 68 | 21 | 270 |
| Classical PTC | 68 | 21 | 247 |
| Folicular variant | | | 16 |
| Tall cell variant | | | 3 |
| Hob [symbol] variant | | | 2 |
| Clear cell variant | | | 1 |
| PTC with fasc [symbol] tis-like | | | 1 |
| st [symbol] e | | | |
| FTC | 14 | 3 | [symbol] |
| PDTC | | | 1 |
| MTC | | | 1 |

[symbol]

indicates data missing or illegible when filed

Abbreviations used in Table 1: ACR TI-RADS, American College of Radiology Thyroid Imaging. Reporting. and Data System; FNA, fine-needle aspiration; FTA, follicular thyroid adenoma; FTC, follicular thyroid carcinoma; HT, Hashimoto's thyroiditis: IQR, interquartile range; MTC, medullary thyroid carcinoma; NG, nodular goiter; PDTC, poorly differentiated thyroid carcinoma; PTC, papillary thyroid carcinoma. aEight benign FNA samples from eight Bethesda II thyroid nodules that were not operated but stayed ultrasonographically stable over a 1-year follow-up were included as benign samples for model optimization but were excluded from specificity calculation. bThe model validation included two patients each with two sets of FNA samples from two different thyroid nodules, resulting in 394 nodules analyzed from 392 patients.

All cytopathologic and histopathologic diagnoses were from a central review by an independent committee of three experienced thyroid pathologists who were blinded to the QCIGISH results. This study was approved by the ethics committees of Shanghai Tenth People's Hospital, Nanjing First Hospital, and Cancer Hospital of the University of Chinese Academy of Sciences (approval number SHSY-IEC-4.1/19-6/01), Jiangyuan Hospital (approval number YL201811), Taizhou People's Hospital and Taizhou Third People's Hospital (approval number TZ20190520), Shengjing Hospital (approval number 2020PS377K), and Henan Cancer Hospital (approval number 2019122504). All participants were age older than 18 years and provided informed consents. This study was registered in the Chinese Clinical Trial Registry (registration number: ChiCTR1900025265).

### Sample Preparation and QCIGISH Detection

For model building, surgical tissues were prepared using a previously described procedure as described in (Shen R, Cheng T, Xu C, et al.: Novel visualized quantitative epigenetic imprinted gene biomarkers diagnose the malignancy of ten cancer types. Clin Epigenetics 12:71, 2020, referred to herein as Shen 2020). Briefly, Tissue blocks were prepared by standard FFPE sample preparation protocol following the RNAscope (Advanced Cell Diagnostics, ACD Bio, Newark, CA, USA) sample preparation procedures and serially cut into 10-μm sections. The sections were then mounted on positively charged slides and dried overnight at room temperature (RT). The sections were deparaffinized in xylene and pretreated following the RNAscope sample preparation procedures. (Wang F, Flanagan J, Su N, Wang L C, Bui S, Nielson A, Wu X, Vo H T, Ma X J, Luo Y. RNAscope: a novel in situ RNA analysis platform for formalin-fixed, paraffin-embedded tissues. The Journal of molecular diagnostics: JMD. 2012; 14 (1): 22-9, referred to herein as Wang 2012). Fine-needle aspiration samples from thyroid samples were fixed immediately after sampling in 10% NBF (neutral buffered formalin) for 48 h at RT. The samples were directly mounted onto positively charged slides and pretreated following the RNAscope sample preparation procedures. Specific probes were designed according to the imprinted genes. The probes were designed according to imprinted genes SNRPN and HM13. Specifically, a sequence within an intron of each gene was selected for probe design. Specific probes were designed by Advanced Cell Diagnostics. RNASCope in situ hybridization was performed on the samples using the probes according to the protocol of the kit. The sections were stained with hematoxylin-eosin stain. The expressions of imprinted genes were analyzed through microscope images.

The total expression (TE) of each of the imprinted genes, the biallelic expression (BAE) of each of the imprinted genes, and the multiallelic expression (MAE) of each of the imprinted genes are calculated by the following formula:

$$TE = ( b + c + d ) / ( a + b + c + d ) \times 100 \% ;$$

$$BAE = c / ( b + c + d ) \times 100 \% ;$$

$$MAE = d / ( b + c + d ) \times 100 \% ;$$

wherein, a represents the number of cells each of which has no mark in the nucleus of the cell after the staining, b represents the number of cells for each of which there is one red/brown mark in the nucleus of the cell after the staining, c represents the number of cells for each of which there are two red/brown marks in the nucleus of the cell after the staining, and d represents the number of cells for each of which there are more than two red/brown markers in the nucleus of the cell after the staining.

For model optimization and blinded prospective validation, a thyroid FNA specimen was divided into two parts for simultaneous cytopathology evaluation and blinded QCIGISH testing. The FNA specimens for QCIGISH testing were fixed in 10% formalin neutral buffer immediately after sampling and were mechanically separated before being mounted on positively charged slides. Each FNA sample was mounted in a well of 10-mm² area with hydrophobic barrier and dried overnight at 60° C.

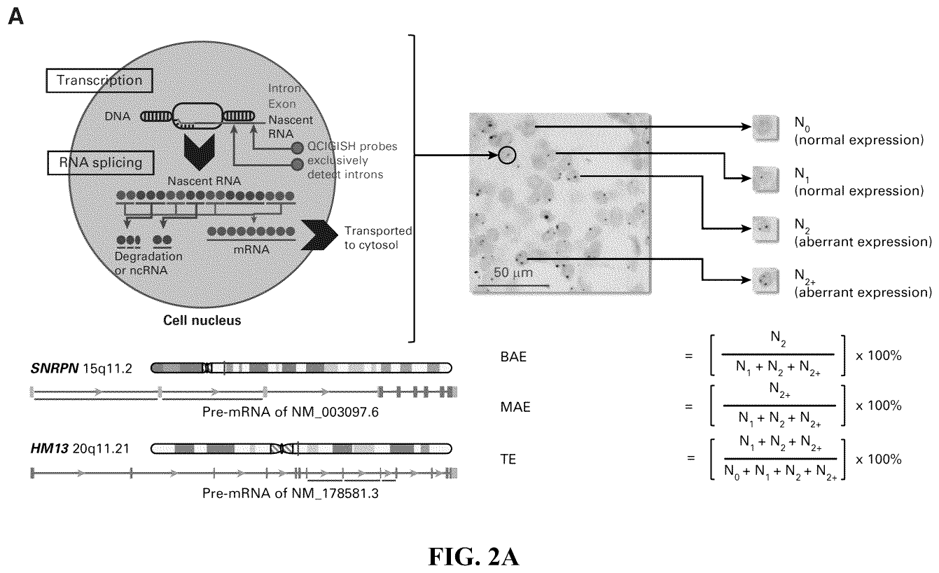

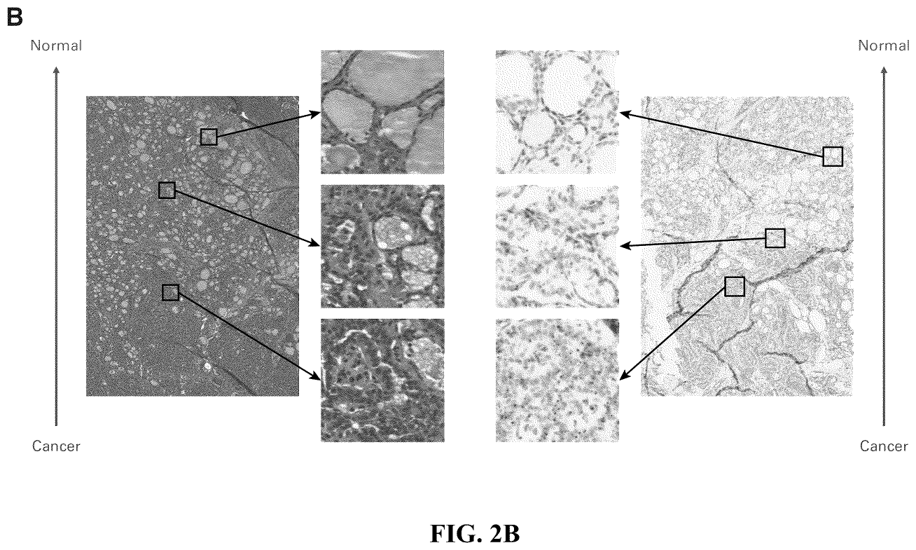

For ISH, the sample slides were pretreated following the RNAscope sample preparation procedures (Wang 2012). ISH was performed as described previously (Shen 2020) using probes targeting the noncoding intronic regions of nascent RNAs from GNAS and HM13, as detailed in FIG. 2A.

The detected gene-expressing site appeared as a distinct red or brown dot under common bright-field microscope (FIG. 2A). For surgical tissue samples, four representative 400× high-power fields were selected for nuclei counting. For FNA specimens, scanned microscopic images with at least 1,200 nuclei for each sample were randomly selected for nuclei counting. BAE, MAE, and TE were determined as discussed herein (see also FIG. 2A).

Each of the imprinted gene HM13 and SNRPN is then graded according to the calculated total expression, biallelic expression, and multiallelic expression. As shown in FIG. 5A and Table 3, for thyroid FNA specimens, for the imprinted gene HM13, if the total expression of HM13 is <11.24%, the biallelic expression of HM13 and the multiallelic expression of HM13 are both determined to be Grade 0; if the total expression is >=11.24%, the biallelic expression of HM13 is classified into 5 grades, and the multiallelic expression of HM13 is also classified into 5 grades: Grade 0 is defined as the biallelic expression of HM13 is <12.39% and the multiallelic expression of HM13 is <1.43%; Grade 1 is defined as the biallelic expression of HM13 is >=12.39 and <20.07%, and the multiallelic expression of HM13 is >=1.43 and <3.39%; Grade 2 is defined as the biallelic expression of HM13 is >=20.07 and <26.28%, and the multiallelic expression of HM13 is >=3.39 and <6.31%; Grade 3 is defined as the biallelic expression of HM13 is >=26.28 and <30.00% and the multiallelic expression of HM13 is >=6.31 and <9.42%; and Grade 4 is defined as the biallelic expression of HM13 is >=30.00% and the multiallelic expression of HM13 is >=9.42%. For the imprinted gene SNRPN, if the total expression of SNRPN is <17.38%, the biallelic expression of SNRPN and the multiallelic expression of SNRPN are both determined to be Grade 0; if the total expression is >=17.38%, the biallelic expression of SNRPN is classified into 5 grades, and the multiallelic expression of SNRPN is also classified into 5 grades: Grade 0 is defined as the biallelic expression of SNRPN is <15.29% and the multiallelic expression of SNRPN is <1.44%; Grade 1 is defined as the biallelic expression of SNRPN is >=15.29 and <20.25%, and the multiallelic expression of SNRPN is >=1.44 and <3.41%; Grade 2 is defined as the biallelic expression of SNRPN is >=20.25 and <26.41%, and the multiallelic expression of SNRPN is >=3.41 and <5.54%; Grade 3 is defined as the biallelic expression of SNRPN is >=26.41 and <30.77% and the multiallelic expression of SNRPN is >=5.54 and <9.02%; and Grade 4 is defined as the biallelic expression of SNRPN is >=30.77% and the multiallelic expression of SNRPN is >=9.02%.

As shown in FIG. 5B, an overall gene score is derived for each imprinted gene according to the grade of the gene's biallelic expression and the grade of the gene's multiallelic expression. Score 0: if the grade of the gene's biallelic expression and the grade of the gene's multiallelic expression are both grade 0; Score 1: if the grade of the gene's multiallelic expression is grade 0 and the grade of the gene's biallelic expression is >=grade 1, or if the grade of the gene's multiallelic expression is grade 1 and the grade of the gene's biallelic expression is <=grade 1; Score 2: if the grade of the gene's multiallelic expression is grade 1 and the grade of the gene's biallelic expression is >=grade 2, or if the grade of the gene's multiallelic expression is grade 2 and the grade of the gene's biallelic expression is <=grade 2; Score 3: if the grade of the gene's multiallelic expression is grade 2 and the grade of the gene's biallelic expression is >=grade 3, or if the grade of the gene's multiallelic expression is grade 3 and the grade of the gene's biallelic expression is <=grade 3; Score 4: if the grade of the gene's multiallelic expression is grade 3 and the grade of the gene's biallelic expression is grade 4, or if the grade of the gene's multiallelic expression is grade 4 and the grade of the gene's biallelic expression is <=grade 4.

As shown in FIG. 5C, the level of malignancy of the thyroid tumor was determined based on an evaluation of the derived overall SNRPN gene score and the derived overall HM13 gene score. The evaluation includes calculating a weighted sum of the derived overall SNRPN gene score and the derived overall HM13 gene score; and comparing the weighted sum against predetermined four numerical thresholds defining five numerical ranges and determining which of the five numerical ranges the weighted sum falls into. A weight of 0.7 is assigned to the overall HM13 gene score whereas a weight of 0.3 is assigned to the overall SNRPN gene score. In some embodiments, the four numerical thresholds are 0.5, 1.5, 2.5, and 3.5, respectively.

### Gene Screening Study, Diagnostic Grading Model Building, and Model Optimization

We determined that SNRPN and HM13 as efficient thyroid cancer biomarkers. This two-gene combination achieved optimally high sensitivity with minimal compromise in specificity. QCIGISH test of the two genes was applied to the model training set of 124 surgical thyroid tissue samples, with the development of a five-grade thyroid cancer prediction model. We showed a correlation between the allelic expression signal numbers of these genes and the morphologic malignancy level by comparing the QCIGISH and hematoxylin and cosin staining on serial tissue sections of the tumor (FIG. 2B).

Grade 0 indicated a benign result while Grade 1 suggested a possible but low malignancy potential, with both being classified as QCIGISH-negative. Grades 2, 3 and 4 were all considered QCIGISH-positive, indicating low, moderate and high malignancy risks, respectively. Taking grades 0 and 1 as negative predictions and Grades 2, 3, and 4 as positive predictions, this model demonstrated an optimism-corrected 93.9% sensitivity (95% CI, 93.6 to 94.1) and 86.3% specificity (95% CI, 86.0 to 86.6).

We independently applied this QCIGISH model established on FFPE to 32 FNA specimens for optimization in presurgical diagnosis and achieved an optimism-corrected sensitivity of 88.6% (95% CI, 88.2 to 89.0) and an optimism-corrected false-positive rate of 1.9% (95% CI, 1.3 to 2.4).

### Statistical Analysis

Continuous variables were reported as medians with interquartile ranges, while frequencies and proportions were reported for categorical variables. For the imprinted gene panel from the model building set, a robust rank-order nonparametric test was used for the comparison of the benign and malignant groups. Area under the curve was used to evaluate and compare the discrimination performance of the imprinted gene panel for the gene screening, model building, and optimization sets. All computed area under the curve values were generated using the ROCR package in R. Hypothesis testing was done in a two-sided manner, with computed P<0.05 considered to be significant. Sensitivity, specificity, positive predictive value (PPV), negative predictive value (NPV), diagnostic accuracy, and 95% CIs were calculated using standard methods. All statistical analyses and visualizations were conducted using R software (version 3.5.0).

### Results

### Test of the Clinical Applicability of QCIGISH in a Blinded Prospective Validation Cohort

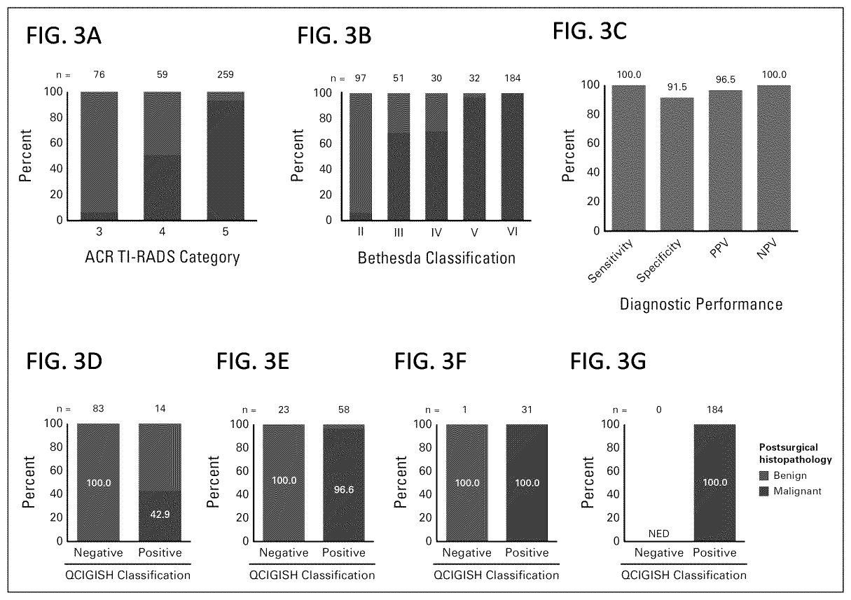

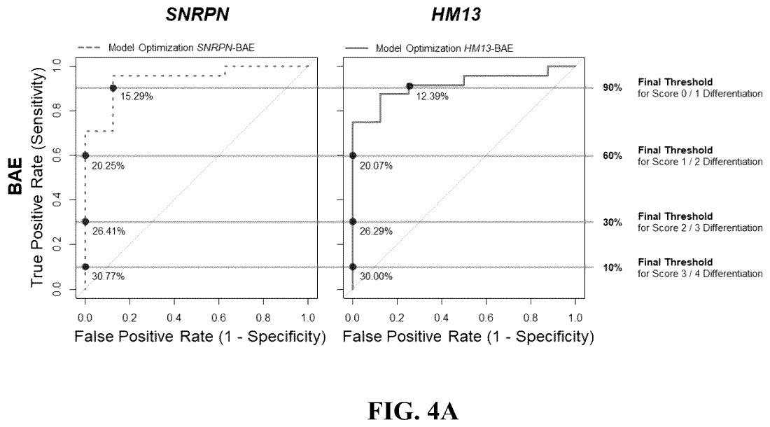

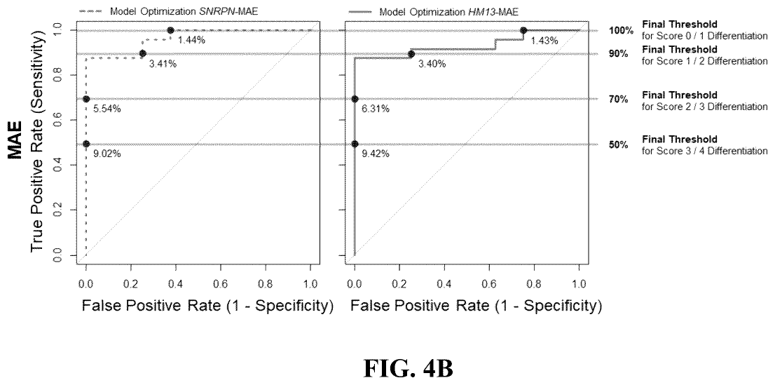

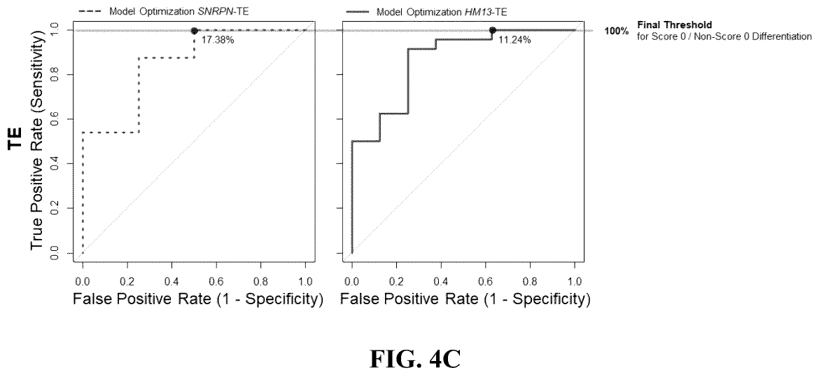

We tested the clinical applicability of the QCIGISH model in an independent blinded prospective validation set of 394 thyroid nodules. An increasing histopathologic malignancy rate was observed from ACR TI-RADS categories 3-5 on ultrasonography (FIG. 3A). The histopathologic malignancy rate was also increased from Bethesda II to Bethesda VI thyroid nodules, as can be expected (FIG. 3B). Using histopathologic diagnoses as the diagnostic reference standard, the final QCIGISH grading model on FNA specimen testing achieved an overall diagnostic sensitivity of 100% (277/277), a specificity of 91.5% (107/117; 95% CI, 86.4 to 96.5), a PPV of 96.5% (95% CI, 94.4 to 98.6), and a NPV of 100% for malignancy in the validation set of 394 nodules (Table 2, FIG. 3C). The corresponding overall diagnostic accuracy was 97.5% (384/394; 95% CI, 95.9 to 99.0).

TABLE 2
Performance of Quantitative Chromo [symbol] nic imprinted Gene [symbol] tion Test
on Fine-Needle Aspiration Specimens in Different Bethesda
Categories of Thyroid Nodules [symbol] the P [symbol] Model V

[symbol]

| | | | |
|---|---|---|---|
| Pathologic Diagnosis | Malignant | Benign | Test Performance, % (95% CI) |

Performance in Bethesda II Nodules (n = 97, malignancy rate 6.2%)

| | | | |
|---|---|---|---|
| Test positive | 6 | 8 | Sensitivity, 100.0 |
| Test negative | 0 | 83 | Specificity, 91.2 (65.4 to 97.0) |
| | | | PPV, 42. [symbol] (16.9 to 68.8) |
| | | | NPV, 100.0 |

Performance in Bethesda III Nodules (n = 51, malignancy rate 68.6%)

| | | | |
|---|---|---|---|
| Test positive | 35 | 1 | Sensitivity, 100.0 |
| Test negative | 0 | 15 | Specificity, 93.8 (81.9 to 100.0) |
| | | | PPV, 97.2 (91.9 to 100.0) |
| | | | NPV, 100.0 |

Performance in Bethesda IV Nodules (n = 30, malignancy rate 70.0%)

| | | | |
|---|---|---|---|
| Test positive | 21 | 1 | Sensitivity, 100.0 |
| Test negative | 0 | 8 | Specificity, 88.9 (69.4 to 100.0) |
| | | | PPV, 9 [symbol] (86.8 to 100.0) |
| | | | NPV, 100.0 |

Performance in Bethesda V Nodules (n = 32, malignancy rate 96.9%)

| | | | |
|---|---|---|---|
| Test positive | [symbol] | 0 | Sensitivity, 100.0 |
| Test negative | 0 | 1 | Specificity, 100.0 |
| | | | PPV, 100.0 |
| | | | NPV, 100.0 |

Performance in Bethesda VI Nodules (n = 184, malignancy rate 100.0%)

| | | | |
|---|---|---|---|
| Test positive | 1 [symbol] 4 | 0 | Sensitivity, 100.0 |
| Test negative | 0 | 0 | Specificity, NED |
| | | | PPV, 100.0 |
| | | | NPV, NED |

Performance Across the Entire Cohort (n = 394, malignancy rate 70.3%)

| | | | |
|---|---|---|---|
| Test positive | 277 | 10 | Sensitivity, 100.0 |
| Test negative | 0 | 107 | Specificity, 91.5 (66.4 to 96.5) |
| | | | PPV, 96.5 (94.4 to 98.6) |
| | | | NPV, 100.0 |

Performance in Bethesda III & IV Nodules (n = 81, malignancy rate 69.1%)

| | | | |
|---|---|---|---|
| Test positive | [symbol] 6 | [symbol] | Sensitivity, 100.0 |
| Test negative | 0 | 23 | Specificity, 92.0 (81.4 to 100.0) |
| | | | PPV, 96.6 (91.9 to 100.0) |
| | | | NPV, 100.0 |

Performance in Bethesda III & V Nodules (n = 113, malignancy rate 77.0%)

| | | | |
|---|---|---|---|
| Test positive | 87 | 2 | Sensitivity, 100.0 |
| Test negative | 0 | 24 | Specificity, 92.3 (82.1 to 100.0) |
| | | | PPV, 97.8 (94.7 to 100.0) |
| | | | NPV, 100.0 |

Abbreviations:
NED, no evidence of disease,
NPV, negative predictive value,
PPV, positive predictive value. [symbol]
indicates data missing or illegible when filed

### The Diagnostic Values of QCIGISH in Different Bethesda Cytologic Categories

The diagnostic performance of the QCIGISH test in different Bethesda cytologic categories is summarized in Table 2. For Bethesda II thyroid nodules, 100% (83/83) of QCIGISH-negative cases were histopathologically benign, while 42.9% (6/14) of QCIGISH-positive cases were histopathologically proven to be malignant (Table, FIG. 3D). For Bethesda III and IV thyroid nodules, 100% (23/23) of QCIGISH-negative cases on FNA specimen testing were histopathologically proven to be benign, while 96.6% (56/58) of QCIGISH-positive cases on FNA specimen testing were histopathologically proven to be malignant, demonstrating a PPV of 96.6% (95% CI, 91.9 to 100) and a NPV of 100%, with a diagnostic accuracy of 97.5% (79/81; 95% CI, 94.2 to 100) for diagnosing the malignancy of thyroid nodules (Table 2, FIG. 3E). For Bethesda V thyroid nodules, the only case classified as QCIGISH-negative was histopathologically proven to be benign, and 100% (31/31) of QCIGISH-positive cases on FNA specimens proved to be histopathologically malignant (Table 2, FIG. 3F). For Bethesda VI thyroid nodules, 100% (184/184) of QCIGISH-positive cases on FNA specimens proved to be histopathologically malignant (Table 2, FIG. 3G). When cytologically indeterminate Bethesda III, IV, and V were combined, 92.3% (24/26; 95% CI, 82.1 to 100) of the FNA cases that were histopathologically proven to be benign were negative on QCIGISH test, and 100% (87/87) of the FNA cases that were histopathologically proven to be malignant were positive on QCIGISH test, demonstrating a PPV of 97.8% (95% CI, 94.7 to 100) and a NPV of 100% for diagnosing the malignancy of thyroid nodules (Table 2). The overall diagnostic accuracy for combined Bethesda III, IV, and V cases was 98.2% (111/113; 95% CI, 95.8 to 100).

In two cases, presurgical QCIGISH test on two nodules in the same patient distinguished the malignant from the benign one, which were histopathologically confirmed. In addition, 52 Bethesda II cases of thyroid nodules not surgically operated were all negative on QCGISH testing.

As a case illustration of the excellent diagnostic performance of QCIGISH test on indeterminate Bethesda thyroid nodules, data show histopathologically confirmed one benign and one malignant thyroid nodule, which were both ultrasonographically ACR TI-RADS category 5 and cytologically Bethesda III and were indeterminate nodules. QCIGISH test was able to presurgically distinguish the benign from the malignant case as histopathologically confirmed.

### DISCUSSION

Although combined ultrasonographic and cytologic evaluation with FNA is currently the diagnostic mainstay for thyroid nodules, it can be challenging, particularly in the case of indeterminate cytology. This is true even with several currently used molecular diagnostic systems, as they each have limitations. In this study, we tested the value of the imprinted gene-based QCIGISH in diagnosing thyroid nodules and demonstrated an excellent diagnostic performance, including a high PPV of 97.8% and a NPV of 100% in cytologically indeterminate Bethesda III-V thyroid nodules. For Bethesda III and IV thyroid nodules, which are most challenging diagnostically, QCIGISH demonstrated a PPV of 96.6% and a NPV of 100%. The high NPV of QCIGISH can effectively help rule out malignancy of thyroid nodules, while its high PPV makes it also an effective rule-in test.

Aberrant expressing status of an imprinted gene often occurs at an early stage of carcinogenesis. An efficient and practical detection method to quantify the imprinting changes to reliably assess malignancy has been lacking. We have recently developed the novel QCIGISH method targeting noncoding intronic nascent RNAs to directly visualize transcription loci of imprinted genes in cell nuclei (Shen 2020). The BAE observed with this method most likely represents LOI, with activation of both the paternal and maternal alleles, but it could also be from the copy-number variation (CNV) of the normally activated allele. Similarly, MAE could represent activation of the normally imprinted allele plus its CNV or just CNV. Our previous studies have demonstrated that BAE, MAE, and TE indeed represented the combined LOI and CNV of imprinted genes in various cancers. Regardless of the mechanism, an increase in the expression signals of the imprinted gene suggests malignancy. We have previously identified three common imprinted genes GNAS, GRB10, and SNRPN showing aberrant expression in 10 cancer types. In this study, we investigated the diagnostic value of these genes and additionally also a novel imprinted gene HM13 in thyroid nodules. Through model building and optimization, we demonstrated that the combination of SNRPN and HM13 had the most efficient diagnostic value that could effectively improve the presurgical diagnosis of thyroid nodules. Following the model optimization using FNA specimens, the prospective validation study with the grading model demonstrated a high diagnostic performance across all thyroid nodules, including Bethesda III-V nodules, which account for about 30% of thyroid nodules, representing a considerable diagnostic challenge in clinical thyroidology. There is well-known diagnostic variability in thyroid cytopathology, especially in indeterminate categories. To be consistent with this, there were several histopathologically confirmed malignant cases of cytologically Bethesda II thyroid nodules in this study. The QCIGISH can now effectively help mitigate this challenge as its diagnostic accuracy is remarkably high across all thyroid nodules regardless of the cytologic categories.

In this study, the malignancy rate in the Bethesda II cases was higher than reported.⁷ This is likely because many of these surgically treated Bethesda II thyroid nodules were clinically symptomatic and might thus have an intrinsically increased malignancy risk. The malignancy rates of Bethesda III and IV thyroid nodules in this study were also relatively high. This was likely because of the use of high-risk ultrasonographic characteristics or BRAF mutation to guide treatment of Bethesda III and IV nodules toward surgery in the hospitals participating in this study. Regardless, QCIGISH demonstrated a robust diagnostic performance across all Bethesda categories. Interestingly, among the three cases of QCIGISH-positive indeterminate histopathology, one case showed BRAF V600E mutation, the second showed double immunohistochemistry staining for CK19 and Hbme-1, and the third showed double immunohistochemistry staining for CK19 and Galectin-3. These molecular markers are known to suggest carcinogenesis or early-stage cancer. As epigenetic alterations occur even before malignant morphologic changes, such apparently false QCIGISH-positive cases might actually have malignant potential and warrant careful clinical follow-up.

The QCIGISH test demonstrated a diagnostic sensitivity and NPV of both 100% in all Bethesda categories of the large cohort of thyroid nodules in this study. As such, this test can effectively help avoid unnecessary thyroid surgeries for benign nodules that are negative on presurgical QCIGISH test but are otherwise cytologically indeterminate. It is remarkable that the QCIGISH test showed such a high diagnostic performance using only two imprinted genes, making it an economically favorable test. Moreover, QCIGISH is based on ISH, which offers an easy and inexpensive yet accurate and robust diagnostic test. The technical simplicity of this test makes it widely applicable practically, which is different than some other molecular tests that are often too complex or too specialized technically to be widely applicable.

The malignant cases of thyroid nodules consisted of mainly classical PTC and some FTC in this study, with some cases of nonclassical PTC. There was only one case of medullary thyroid carcinoma. Consistent with their rarity in the Asian population, Hürthle cell adenoma and carcinoma and noninvasive follicular thyroid neoplasm with papillary-like nuclear features were not included in this study. Also, the oncogenic functionality, if any, of imprinted genes SNRPN and HM13 in the thyroid gland is not well known, making it only speculative to understand their functional relevance with respect to their high diagnostic performance for thyroid malignancy.

In summary, to our knowledge, this study for the first time demonstrates that the imprinted gene-based QCIGISH test has a robust diagnostic performance for thyroid nodules. Its high NPV makes this test highly effective in ruling out malignancy, while its high PPV makes it also an excellent rule-in test, which will be particularly helpful in assisting the evaluation of cytologically indeterminate thyroid nodules. As such, this novel thyroid molecular diagnostic test will likely have a significant clinical impact.

All publications, patents and patent applications referred to herein are incorporated by reference in their entirety to the same extent as if each individual publication, patent or patent application was specifically and individually indicated to be incorporated by reference in its entirety. In the event of inconsistent usages between this document and those documents so incorporated by reference, the usage in the incorporated reference(s) should be considered supplementary to that of this document; for irreconcilable inconsistencies, the usage in this document controls.

The present application illustrates the detailed methods of the present invention by the above examples, but not limited to the above detailed examples, that is, it does not mean that the present invention must rely on the detailed methods described above to be implemented. It should be apparent to those skilled in the art that any modifications of the present application, the equivalent replacement of each raw material of the products of the present application, the addition of an auxiliary component, the selection of a specific manner, and the like, are all within the scope of protection and disclosure of the present application.

1. A method for determining a level of malignancy of a thyroid tumor in a subject and treating the subject, comprising:
obtaining a test sample from the subject;
performing in situ hybridization of a first probe designed based on the sequence of an intron of imprinted gene HM13 with a first plurality of cells of the test sample, and performing in situ hybridization of a second probe designed based on the sequence of an intron of imprinted gene SNRPN with a second plurality of cells of the test sample;
staining the first plurality of cells and the second plurality of cells having been subject to the hybridization with a staining chemical,
calculating a total expression, a biallelic expression, and a multiallelic expression for imprinted genes HM13 based on microscopic images of the stained first plurality of cells, and calculating a total expression, a biallelic expression, and a multiallelic expression for imprinted genes SNRPN based on microscopic images of the stained second plurality of cells, respectively;
grading the biallelic expression and multiallelic expression of each of the imprinted genes HM13 and SNRPN, deriving an overall gene score for each of the imprinted genes HM13 and SNRPN, and determining the level of malignancy of a thyroid tumor based on a combination of the overall gene score for imprinted gene HM13 and imprinted gene SNRPN; and
treating the subject by administration of medication or other treatment in accordance with the determined level of malignancy of said thyroid tumor;
wherein the total expression (TE) of each of the imprinted genes HM13 and SNRPN, the biallelic expression (BAE) of each of the imprinted genes HM13 and SNRPN, and the multiallelic expression (MAE) of each of the imprinted genes HM13 and SNRPN are calculated by the following formula:
$$TE = ( b + c + d ) / ( a + b + c + d ) \times 100 \% ;$$
$$BAE = c / ( b + c + d ) \times 100 \% ;$$
$$MAE = d / ( b + c + d ) \times 100 \% ;$$
wherein, a represents the number of microscopically observed cells each of which has no mark in the nucleus of the cell after the staining, b represents the number of microscopically observed cells for each of which there is one red/brown mark in the nucleus of the cell after the staining, c represents the number of microscopically observed cells for each of which there are two red/brown marks in the nucleus of the cell after the staining, and d represents the number of microscopically observed cells for each of which there are more than two red/brown markers in the nucleus of the cell after the staining.

2. The method according to claim 1, wherein the staining chemical comprises hematoxylin.

3. The method according to claim 1, wherein the biallelic expression of each of the imprinted genes and the multiallelic expression of each of the imprinted genes is classified into 5 grades.

4. The method according to claim 3, wherein if the total expression of HM13 is smaller than a first predetermined threshold T_{Z16-T1}, the biallelic expression of HM13 and the multiallelic expression of HM13 are both classified as Grade 0, and if the total expression of HM13 is greater or equal to T_{Z16-T1}, the biallelic expression of HM13 is classified into 5 grades according to four thresholds T_{Z19-B1}, T_{Z19-B2}, T_{Z19-B3}, T_{Z19-B4}, and the multiallelic expression of HM13 is also classified into the following five grades according to four thresholds T_{Z19-M1}, T_{Z19-M2}, T_{Z19-M3}, T_{Z19-M4}:
Grade 0: the biallelic expression of HM13 is <T_{Z19-B1} and the multiallelic expression of HM13 is <T_{Z19-M1};
Grade 1: the biallelic expression of HM13 is >=T_{Z19-B1} and <T_{Z19-B2}, and the multiallelic expression of HM13 is >=T_{Z19-M1} and <T_{Z19-M2};
Grade 2: the biallelic expression of HM13 is >=T_{Z19-B2} and <T_{Z19-B3}, and the multiallelic expression of HM13 with is >=T_{Z19-M2} and <T_{Z19-M3};
Grade 3: the biallelic expression of HM13 is >=T_{Z19-B3} and <T_{Z19-B4}, and the multiallelic expression of HM13 is >=T_{Z19-M3} and <T_{Z19-M4};
Grade 4: the biallelic expression of HM13 is >=T_{Z19-B4} and the multiallelic expression of HM13 is >=T_{Z19-M4}.

5. The method according to claim 4, wherein if the total expression of SNRPN is smaller than a second predetermined threshold T_{Z19-T1}, the biallelic expression of SNRPN and the multiallelic expression of SNRPN are both classified as Grade 0, and if the total expression of SNRPN is greater or equal to T_{Z19-T1}, the biallelic expression of SNRPN is classified into 5 grades according to four thresholds T_{Z16-B1}, T_{Z16-B2}, T_{Z16-B3}, T_{Z16-B4}, and the multiallelic expression of SNRPN is also classified into the following five grades according to four thresholds T_{Z16-M1}, T_{Z16-M2}, T_{Z16-M3}, T_{Z16-M4}:
Grade 0: the biallelic expression of SNRPN with is <T_{Z16-B1} and the multiallelic expression of SNRPN is <T_{Z16-M1};
Grade 1: the biallelic expression of SNRPN with is >=T_{Z16-B1} and <T_{Z16-B2}, and the multiallelic expression of SNRPN is >=T_{Z16-M1} and <T_{Z16-M2};
Grade 2: the biallelic expression of SNRPN with is >=T_{Z19-B2} and <T_{Z16-B3}, and the multiallelic expression of SNRPN is >=T_{Z16-M2} and <T_{Z16-M3};
Grade 3: the biallelic expression of SNRPN with is >=T_{Z16-B3} and <T_{Z16-B4}, and the multiallelic expression of SNRPN is >=T_{Z16-M3} and <T_{Z16-M4};
Grade 4: the biallelic expression of SNRPN with is >=T_{Z16-B4} and the multiallelic expression of SNRPN with is >=T_{Z16-M4}.

6. The method according to claim 5, further comprising:
deriving an overall gene score for SNRPN based on the determined grade of biallelic expression of SNRPN and the determined grade of multiallelic expression of SNRPN; and
deriving an overall gene score for HM13 based on the determined grade of biallelic expression of HM13 and the determined grade of multiallelic expression of HM13.

7. The method according to claim 6, wherein the level of malignancy of the thyroid tumor is determined based on an evaluation of both the overall gene score of SNRPN and the overall gene score of HM13.

8. The method according to claim 7, wherein the level of malignancy of the thyroid tumor to be determined is classified as 5 categories indicated by integers 0, 1, 2, 3, and 4, wherein greater integers indicate greater malignancy, with 0 indicating benign thyroid tumor, and 4 indicating thyroid tumor with the highest malignancy.

9. The method according to claim 8, wherein the evaluation comprises:
calculating a weighted sum of the overall SNRPN gene score and the overall HM13 gene score; and
comparing the weighted sum against a set of predetermined four numerical thresholds defining five numerical ranges and determining into which of the five numerical ranges the weighted sum falls.

10. The method according to claim 9, wherein a greater weight is assigned to the overall HM13 gene score.

11. The method of any of claims 4-10, wherein
T_{Z19-T1}=11.24%;
T_{Z19-B1}, T_{Z19-B2}, T_{Z19-B3}, T_{Z19-B4}=12.39%, 20.07%, 26.28%, and 30.00%, respectively; and
T_{Z19-M1}, T_{Z19-M2}, T_{Z19-M3}, T_{Z19-M4}=1.43%, 3.39%, 6.31%, and 9.42%, respectively.

12. The method of any of claims 5-11, wherein
T_{Z16-T1}=17.38%;
T_{Z16-B1}, T_{Z16-B2}, T_{Z16-B3}, TZ16−84=15.29%, 20.25%, 26.41%, and 30.77%, respectively; and
T_{Z16-M1}, T_{Z16-M2}, T_{Z16-M3}, T_{Z16-M4}=1.44%, 3.41%, 5.54%, and 9.02%, respectively.

13. The method of claim 9, wherein the four numerical thresholds are 0.5, 1.5, 2.5, and 3.5, respectively.

14. The method according to any of the foregoing claims, wherein the test sample is human tissue and/or cells.

15. The method according to any of the foregoing claims, wherein the test sample is a needle biopsy sample.

16. The method according to any of the foregoing claims, wherein the in situ hybridization is RNAscope in situ hybridization.
